(19) United States
(12) Patent Application Publication
(10) Pub. No.: US 2014/0271814 A1
(43) Pub. Date: Sep. 18, 2014

(54) CELL BINDING PEPTIDE DRUG DELIVERY SYSTEM AND COMPOUND FOR TREATING CANCER AND TUMORS

(71) Applicant: Shiva Andrali, Los Angeles, CA (US)

(72) Inventor: Shiva Andrali, Los Angeles, CA (US)

(21) Appl. No.: 13/826,120

(22) Filed: Mar. 14, 2013

(51) Int. Cl. A61K 9/127; A61K 33/24; A61K 31/704; A61K 31/7068

(52) U.S. Cl. CPC A61K 9/1271; A61K 31/7068; A61K 33/24; A61K 31/704
USPC 424/450; 514/44 R; 514/19.3; 514/7.6; 514/44 A; 424/174.1; 514/169; 514/9.7; 424/725; 514/49; 424/649; 514/34

(57) ABSTRACT

The present invention is a method and compound for treating cancer and tumors. The invention treats cancer by encapsulating certain cancer fighting drugs in a liposome. Peptides attached to the compound then guide the compound towards its target. During its journey, DSPE-PEG is used to stabilize the compound, to allow more time in the blood stream before losing effectiveness.

### FIELD OF INVENTION

This invention relates to the method and composition for treating cancer and tumors. More particular, the invention relates to encapsulating therapeutic drugs in order to increase stability in the bloodstream and direct the drugs effectively. The composition is easily administered, increases effectiveness, and lowers negative side-effects.

### BACKGROUND

For centuries, people have attempted to cure ailments and diseases with whatever means they had available at the time. Initially, this could include rituals or sacred procedures. As time progressed, people started to discover the efficacy of certain herbs, roots, and other naturally occurring substances in the treatment of ailments. As civilization progressed even further, science allowed for humans to discover what made the herbs and roots so effective. Useful and effective compounds were identified, isolated, purified, and administered with great efficacy in the treatment of diseases.

People then discovered they could actually create compounds, based both on knowledge gleaned from their past in combination with knowledge gained from scientific experimentation. With this new creative ability, diseases were fought on massive scales, and deaths as a result dropped drastically. As of now, two diseases were even fought to eradication, smallpox and rinderpest, and numerous other diseases are believed to be just a few years away from eradication. Yet, a select few diseases have managed to go from a relatively rare, to leading causes of death. In the United States, cancer is currently the second leading killer, just behind heart disease. The third leading killer, chronic lower respiratory disease, kills 75 percent fewer people than cancer. Cancer and heart disease have become by far the deadliest killers in the United States. It is possible that the current rise of cancer is due to living longer, which allows more genetic mutations to build up, but cancer is still a treatable disease.

When cancer was first discovered as a pathogen, there was no real treatment for treating cancer. However, as cancer has become more prevalent and detectable, efforts at fighting and treating cancer increased as well. One approach to fighting cancer is the use of drugs, known as chemotherapy, which in its infancy merely acted as a poison, with the hope that it would kill the cancerous cells before the rest of the body. The rationale for using chemotherapy is that because cancerous cells reproduce more quickly, they would die more quickly if a drug that attacked reproducing cells was used. Chemotherapy clearly has its drawbacks, as it takes a toll on the patient's body, killing many of the patient's cells which reproduce quickly, such as bone marrow, blood cells, and hair cells. In many cases, the drugs are unable to completely eliminate the cancer. Accordingly, a way to specifically target only the cancerous cells and tumors was needed.

method that was, and is still, used to increase efficiency of the treatment is to guide the toxic chemotherapy drugs towards their intended cancerous targets. Eventually, it was determined that certain properties of cancerous cells made them uptake certain compounds at higher rates than the rest of the body.

A helpful discovery was that certain peptides could specifically bind to cancerous cells and tumors due to the presence of specific antigens. Further, research led to the discovery of ways of isolating particularly useful peptides, identifying their functional elements, and finally creating peptides previously unknown to nature in order to seek out specific cells. These targeting advances dramatically increased the specificity of drug delivery systems. Even with all of these advances, it is difficult to identify what the mechanism of action for binding cancerous cells is, and often requires finding the molecular structures of the peptide in interest and whatever cancerous cells' antigens it identifies, both in bound and unbound states.

To increase effectiveness of drugs, methods were discovered to stabilize the drug compounds while in the bloodstream, protecting the drugs from decaying before they reached the cancerous cells or tumors. With knowledge of these general treatment and efficacy principles, the search to discover more effective drugs and methods of stabilization continues.

Liposomes were discovered to be quite versatile, as they can form around desirable molecules, creating a sort of package. Unfortunately, due to their fatty, non-polar nature, liposomes have difficulty staying effective in aqueous solutions, such as blood, which can often result in anything planted in these liposomes not having enough time to make it to their intended target. Even with this limitation, liposomes have offered the enclosed drug increased protection from the patient's body or host system.

Liposomes also allow for easy attachment of additional molecules to the outside of the liposome without modifying the functionality of the drug or molecule contained within the liposomes.

Liposomes have the added effect of reducing the volume of distribution because their size precludes them from entering tissue via healthy capillaries. The liposomes are simply too big to enter the capillaries. However, the capillaries surrounding tumors were discovered to have enhanced permeability and retention, which is commonly referred to as “leaking.” This effectively means that tumor capillaries have larger openings, which liposomes could fit into, and thereby accumulate. This was, and continues to be, a major way in which drugs and other large molecules are directed towards their target, through the inherent properties of the drug size and the cancer cells.

Previous inventions have attempted to combine molecules such as DSPE-PEG (1,2-Distearoyl-sn-Glycero-3-Phosphoethanolamine—Polyethylene Glycol) with various liposomes, with the end goal being to stabilize these artificial vesicles for whatever purpose they may have in the blood stream. The relatively polar nature of the DSPE-PEG attachment allows for liposomes to travel in the bloodstream for longer before failing, which was a major breakthrough in drug based cancer treatments. With the ability to encapsulate molecules in a liposome for longer durations, the possibilities were opened dramatically.

With the ability to protect and guide molecules, it is exceedingly important to identify useful molecules and find useful combinations to guide those molecules. This may have resulted in a double edged sword, as now there are a voluminous amount of useful molecules or suspected useful molecules, but as a consequence, there are virtually an infinite number of combinations using these molecules. Certain molecules or drugs have beneficial effects when combined with other molecules, but without going through rigorous experimentation, often including human trials, many of these combinations are, and will remain, undiscovered. The problem with combining molecules, however, is that the results can be hard to predict, and in many cases can cause more harm than good. Many drugs may be used subsequently without disastrous side-effects, but concurrently taking an effective dose of that same drug can result in serious side-effects, including death. As a result, experimentation must be done slowly and deliberately, and a lot of effort may result in non-useful formulations. Because of this, much of the existing cancer fighting compounds tend to consist of a single effective drug and various stabilizers and targeting components. The cost and danger associated with experimentally combining different drugs in a patient can be both extremely costly, and more importantly, extremely dangerous.

Further, even though it is known that combining different molecules in a single drug treatment compound is theoretically possible, it is not possible to predict with certainty what the combination may do. Because there are a multitude of biochemical pathways in the body, and studying an entire body is too complex and has too many confounding factors, most experiments are done in vitro under conditions that the experimenter believes may be relevant. Once data is acquired, and there is some level of confidence in what the compound actually does, experiments with live patients or other organisms may be started. However, due to the complexity of a fully functioning host, the compound may interfere or react with pathways wholly unaccounted for in the in vitro trials. Even though two molecules appear to act on different biochemical pathways, it is possible that, in combination, they will wholly inhibit a completely different pathway, whereas, when alone, they would only inhibit one part of the pathway which the body could compensate for by using an alternate pathway. Thereby, the pathway in danger of being shut down would be undetected until the two drugs are used in conjunction.

With these and many other discoveries, many breakthroughs have been made, and patients are fighting and living with cancer for longer than ever before. Unfortunately, even with all the knowledge and drugs at our disposal, cancer still kills countless people. This is because the drugs are simply not effective enough to cure cancer, and finding methods of combining drugs to increase effectiveness is extremely slow, costly, and difficult work.

U.S. Published Patent Application No. 2012/0219618, filed by Madsen et al. specifically teaches that a preferred way of using cisplatin is by itself, as mentioned in the preferred embodiment. Additionally, U.S. Published Patent Application No. 2012/0027727, filed by Hall et al. mentions the use of cisplatin in combination with the drugs methotrexate, ifosfamide, and Adriamycin, but fails to describe with any additional specificity what combinations of the unknown infinite combinations are actually useful. Even though Hall mentions the use of cisplatin and gemcitabine in the same patent application, and attempts to disclose several embodiments of multiple drugs used in combination, at no time is there any hint of suggestion to combine cisplatin and gemcitabine.

Thus, there exists the need for effective methods and compounds for treating cancer and tumors.

### SUMMARY OF THE INVENTION

To minimize the limitations in the prior art, and to minimize other limitations that will become apparent upon reading and understanding the present specification, the present invention is a method and compound of treating cancer and tumors, wherein the compound is stabilized and directed to cancer and tumors.

One embodiment of the invention is a cancer treatment compound comprising: one or more therapeutic drugs in an amount of from 8.0 to 48.0 percent by weight based on a total weight of the cancer fighting compound; a liposome component in an amount of from 34.0 to 63.0 percent by weight based on a total weight of the cancer fighting compound; a peptides component in an amount of from 8.0 to 16.0 percent by weight based on a total weight of the cancer fighting compound; a DSPE-PEG component in an amount of from 10.0 to 20.0 percent by weight based on a total weight of the cancer fighting compound; Preferably, the one or more therapeutic drug may be selected from one or more of: gemcitabine, cisplatin, and doxorubicin. Preferably, the peptide may be a Collagen binding peptide, similar to those peptides used in Rexin-G. Preferably, the therapeutic drugs are encapsulated in the liposome. Preferably, the liposome is a phospholipid bilayer. Preferably, the peptide attaches to the liposome, and may cause the cancer treatment compound to preferentially bind to cancerous cells and tumors and to accumulate near cancerous cells and tumors. Preferably, the DSPE-PEG is attached to the peptide, and may stabilize the cancer compound to allow the cancer treatment compound to spend more time in a bloodstream before becoming less effective. Preferably, a single molecule of the cancer treatment compound may contain different therapeutic drugs at the same time.

Another embodiment of the invention is a cancer treatment compound comprising: one or more therapeutic drugs in an amount of from 8.0 to 48.0 percent by weight based on a total weight of the cancer fighting compound; a liposome component in an amount of from 34.0 to 63.0 percent by weight based on a total weight of the cancer fighting compound; a peptides component in an amount of from 8.0 to 16.0 percent by weight based on a total weight of the cancer fighting compound; a DSPE-PEG component in an amount of from 10.0 to 20.0 percent by weight based on a total weight of the cancer fighting compound. Preferably, the one or more therapeutic drugs may be selected from one or more of: gemcitabine, cisplatin, and doxorubicin. Preferably, the peptide may be a Collagen binding peptide, similar to those peptides used in Rexin-G. Preferably, the therapeutic drugs are encapsulated in the liposome. Preferably, the liposome is a phospholipid bilayer. Preferably, the peptide attaches to the liposome, and may cause the cancer treatment compound to preferentially bind to cancerous cells and tumors and to accumulate near cancerous cells and tumors. Preferably, the DSPE-PEG is attached to the peptide, and may stabilize the cancer compound to allow the cancer treatment compound to spend more time in a bloodstream before becoming less effective. Preferably, a single molecule of the cancer treatment compound contains only one type of the therapeutic drugs at a time.

Another embodiment of the invention is a cancer treatment compound comprising: one or more therapeutic drugs in an amount of from 8.0 to 48.0 percent by weight based on a total weight of the cancer fighting compound; a liposome component in an amount of from 34.0 to 63.0 percent by weight based on a total weight of the cancer fighting compound; a peptides component in an amount of from 8.0 to 16.0 percent by weight based on a total weight of the cancer fighting compound; a DSPE-PEG component in an amount of from 10.0 to 20.0 percent by weight based on a total weight of the cancer fighting compound. Preferably, the one or more therapeutic drugs may be doxorubicin. Preferably, the therapeutic drugs are encapsulated in the liposome. Preferably, the liposome is a phospholipid bilayer.

Another embodiment of the invention is a cancer treatment compound comprising: one or more therapeutic drugs in an amount of from 9.0 to 50.0 percent by weight based on a total weight of the cancer fighting compound; a liposome component in an amount of from 38.0 to 70.0 percent by weight based on a total weight of the cancer fighting compound. Preferably, the one or more therapeutic drugs is doxorubicin. Preferably, the therapeutic drugs are encapsulated in the liposome. Preferably, the liposome is a phospholipid bilayer.

Another embodiment is a therapeutic drug, encapsulated in a liposome. Preferably the liposome has a peptide attached to the liposome. Preferably, the peptide also has a DSPE-PEG attached to the peptide.

The Collagen binding peptides are derived from Von Willebrand Factors. Some specific sequence examples of the Collagen binding peptide may include, but are not limited to:

| | |
|---|---|
| Seq1: | Trp-Arg-Glu-Pro-Ser-Phe-Met-Ala-Leu-Ser |
| | (WREPSFMALS) |
| or | |
| Seq2: | Trp-Arg-Glu-Pro-Ser-Phe-Cys-Ala-Leu-Ser |
| | (WREPSFCALS) |

The following sequences are related to the above sequences by one or more conservative amino acid substitutions, which produce substantially the same effect:

| | |
|---|---|
| Seq3: | Trp-Arg-Asp-Pro-Ser-Phe-Met-Ala-Leu-Ser (WRDPSFMALS); |
| Seq4: | Trp-Arg-Asp-Pro-Ser-Phe-Cys-Ala-Leu-Ser (WRDPSFCALS); |
| Seq5: | Trp-Arg-Glu-Pro-Ser-Phe-Met-Ala-Ile-Ser (WREPSFMAIS); |
| Seq6: | Trp-Arg-Glu-Pro-Ser-Phe-Cys-Ala-Leu-Ser (WREPSFCALS); |
| Seq7: | Trp-Arg-Asp-Pro-Ser-Phe-Met-Ala-Leu-Ser (WRDPSFMALS); |
| Seq8: | Trp-Arg-Glu-Pro-Ser-Phe-Cys-Ala-Leu-Ser (WREPSFCALS); |

All of the above peptides may be used in conjunction with a fluorescent dye by addition of Fluorescein isothiocyanate (FITC) to either end of the peptide. The following sequence example shows a peptide which can have the fluorescent dye FITC attached to the amino terminal by addition to PPGP to the front of the original peptide.

| | |
|---|---|
| Seq9: | Pro-Pro-Gly-Pro-Trp-Arg-Glu-Pro-Ser-Phe-Met-Ala-Leu-Ser |
| | (PPGPWREPSFMALS) |

Also, linker peptides may be added on either side of the above sequences:

| | |
|---|---|
| Seq10: | Gly-Pro-Pro-Gly-Trp-Arg-Glu-Pro-Ser-Phe-Met-Ala-Leu-Ser-Gly-Pro-Pro-Gly |
| | (GPPGPWREPSFMALSGPPG) |
| Seq11: | Gly-Pro-Pro-Gly-Trp-Arg-Glu-Pro-Ser-Phe-Cys-Ala-Leu-Ser-Gly-Pro-Pro-Gly |
| | (GPPGPWREPSFCALSGPPG) |
| Seq12: | Gly-Pro-Pro-Gly-Trp-Arg-Asp-Pro-Ser-Phe-Met-Ala-Leu-Ser-Gly-Pro-Pro-Gly |
| | (GPPGPWRDPSFMALSGPPG) |
| Seq13: | Gly-Pro-Pro-Gly-Trp-Arg-Asp-Pro-Ser-Phe-Cys-Ala-Leu-Ser-Gly-Pro-Pro-Gly |
| | (GPPGPWRDPSFMALSGPPG) |
| Seq14: | Gly-Pro-Pro-Gly-Trp-Arg-Glu-Pro-Ser-Phe-Met-Ala-Ile-Ser-Gly-Pro-Pro-Gly |
| | (GPPGPWREPSFMAISGPPG) |
| Seq15: | Gly-Pro-Pro-Gly-Trp-Arg-Glu-Pro-Ser-Phe-Cys-Ala-Ile-Ser-Gly-Pro-Pro-Gly |
| | (GPPGPWREPSFCAISGPPG) |
| Seq16: | Gly-Pro-Pro-Gly-Trp-Arg-Asp-Pro-Ser-Phe-Met-Ala-Ile-Ser-Gly-Pro-Pro-Gly |
| | (GPPGPWRDPSFMAISGPPG) |
| Seq17: | Gly-Pro-Pro-Gly-Trp-Arg-Asp-Pro-Ser-Phe-Cys-Ala-Ile-Ser-Gly-Pro-Pro-Gly |
| | (GPPGPWRDPSFCAISGPPG) |

Further this linker peptide may be used in tandem repeats, for example: [Gly-Pro-Pro-Gly-X1-Gly-Pro-Pro-Gly-X2-Gly-Pro-Pro-Gly]. Where X1 and X2 are one of the peptide sequences from Seq1 through Seq9.

The therapeutic drugs are understood to preferably encompass: anti-neoplastic therapeutic agents, non-steroidal anti-inflammatory therapeutic agents, steroidal anti-inflammatory therapeutic agents, nucleic acid or nucleotide sequence for non-viral gene therapy, hormones, peptides, growth factors, antiangiogenic compounds, antisense oligonucleotides, anti-micro RNA molecules, microRNA molecules, herbal drugs, and antibodies.

The anti-neoplastic therapeutic agents are preferably selected from one or more of: mechlorethamine, cyclophosphamide, ifosmamide, melphalan, chlorambucil, hexamethylmelamine, thiotepa, busulfan, carmustine, streptozotocin, dacarbazine, temozolomide, methotrexate, 5-fluorouracil, cytarabine, gemcitabine, 6-mercaptopurine, 6-thioguanine, pentostatin, vinblastine, vincristine, paclitaxel, docetaxel, topetecan, irinotecan, dactinomycin, daunorubicin, doxorubicin, bleomycin, mitomycin C, L-asparaginase, interferon-alpha, interleukin-2, cisplatin, carboplatin, mitoxantrone, hydroxyurea, N-methylhydrazine, mitotane, aminoglutethimide, imatinib, prednisone, prednisolone, methylprednisolone, dexamethasone, betamethasone, triamcinolone, hydroxyprogesterone, medroxyprogesterone, megestrol acetate, diethylstilbestrol, ethinyl estradiol, tamoxifen, anastrozole, testosterone propionate, fluoxymesterone, flutamine, leuprolide, trastuzumab, rituximab, alemtuzumab, bevacizumab, cetuximab, gemtuzumab, ibritumomab, panitumumab, tositumomab, interferon, Atractylodes lancea, berberine, artemesinin, gossypol, diindolyl methane, emodin, capsaicin, eipgallocatechin gallate, huperzine A, usniacin (D-usnic acid), indolebutyric acid, methylumbelliferone (4-MU), aesculin (Esculin), amygdalin, andrographolide, apigenin, and bilobalide.

The non-steroidal anti-inflammatory drug is preferably selected from one or more of: acetylsalicylic acid (aspirin) sodium salicylate, choline magnesium trisalicylate, salsalate, diflunisal, sulfasalazine, olsalazine, acetaminophen, indomethacin, sulindac, tolmetin, diclofenac, ketorolac, ibuprofen, naprofen, flurbiprofen, ketoprofen, fenoprofin, oxaprozin, mefenamic acid, meclofenamic acid, piroxican, meloxicam, nabumetone, rofecoxib, celecoxib, etodolac, and nimesulide.

The steroidal anti-inflammatory therapeutic is preferably selected from one or more of: hydrocortisone, cortisone, beclomethasone, dipropionate, betamethasone, dexamethasone, prednisone, methylprednisolone, triamcinolone, fluocinolone, acetonide, fludrocortisones, and beclometasone propionate.

The present invention is a cancer fighting compound that may be used to fight a wide variety of cancers and cancer types.

The method the therapeutic drug is administered is preferably intravenously. Preferably this may be done via needle injection or IV drip.

It is an object of the present invention to overcome the limitations of the prior art.

Another object of the present invention is to provide a cancer fighting or treatment compound that is more effective than previous cancer fighting or treatment compounds.

Another object of the present invention is to reduce toxicity of the therapeutic drug treatment by directing the therapeutic drug preferentially towards cancerous cells and tumors by use of the Collagen binding peptides, thereby protecting the non-cancerous cells to some degree.

Another object of the present invention is to reduce the required dosage amounts of therapeutic drug treatments by stabilizing the cancer fighting compound such that the half-life is increased.

Another object of the present invention is to reduce the required dosage amounts of therapeutic drug treatments by directing the drugs towards the targets.

Another object of the present invention is to provide a cancer fighting compound that is stable and easy to prepare.

Another object of the present invention is to provide a treatment which extends the lives of patients with cancer.

Another object of the present invention is to provide patients with better living conditions.

It should be understood that the therapeutic drugs are effective on tumors and cancer. It should be understood that a variety of solvents may be used in preparation of the compound. Preferably, the solvent used has a similar salinity to blood, so as to not cause damage upon injection. One such solvent is 9% saccharose.

Preferably, the solvent used may vary depending on whether a concentrated or a dilute drug composition is desired.

A fluorescent dye may be used in conjunction with the compound. This can be used to easily track the compound.

It should be understood that once the peptide causes the compound to get lodged or bound to a cancerous cell or tumor, the drug is released from the liposome to be taken up by the cancerous cell or tumor.

Additional embodiments of the invention will be understood from the detailed description of the invention.

### BRIEF DESCRIPTION OF THE DRAWINGS

FIG. 1 is a cross-sectional view of one embodiment of a liposome.

### DETAILED DESCRIPTION OF THE INVENTION

In the following detailed description of various embodiments of the invention, numerous specific details are set forth in order to provide a thorough understanding of various aspects of one or more embodiments of the invention. However, one or more embodiments of the invention may be practiced without some or all of these specific details. In other instances, well-known methods, procedures, and/or components have not been described in detail so as not to unnecessarily obscure aspects of embodiments of the invention.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the figures, and the detailed descriptions thereof, are to be regarded as illustrative in nature and not restrictive. Also, the reference or non-reference to a particular embodiment of the invention shall not be interpreted to limit the scope of the invention.

The present invention is aimed at increasing the life expectancy of people with various cancers and tumors by fighting cancerous cells and tumors and causing these cells and tumors to undergo apoptosis at a much higher than normal rate. The compound used selectively targets certain cancers and tumors, effectively decreasing the required dosage of therapeutic drugs and increasing effectiveness.

Table 1 shows a preferred composition of one embodiment of the invention. The ingredients are prepared to create a cancer and tumor fighting compound that is most effective when administered intravenously. The Table also lists the preferred weight (Wt) percentage (%) range of each ingredient and the primary purpose of the ingredient.

TABLE 1

| | Preferred Wt | |
|---|---|---|
| Ingredient | % Range | Purpose |
| Liposome | 34.0 to 64.0 | Improve circulation time of the |
| | | drug, and allow for easier |
| | | combination with other components |
| Therapeutic drug | 8.0 to 48.0 | Fight cancer and tumors |
| (gemcitabine, | | |
| cisplatin, | | |
| doxorubicin, etc.) | | |
| Collagen binding | 8 to 16 | Guide the compound preferentially |
| peptides | | towards cancerous cells and tumors. |
| DSPE-PEG | 10.0 to 20.0 | Stabilizes the entire compound. |

Liposome, as shown in FIG. 1 is preferably used to encapsulate the drug, and to allow for addition of further components without modifying the drug. The liposome also acts to allow for absorption of lipophobic drugs into fatty tissues. Also, because the drug is encapsulated, it is protected from being attacked and broken down by the host. It is relatively simple to attach various peptides and other molecules to the outside of a liposome, without modifying the drug itself. The presence of a liposome allows the Collagen binding peptides to be easily added to the compound, without modifying the functional therapeutic drug. Preferably the liposome component is in an amount up to 70.0 percent by weight, and more preferably between 34.0 to 64.0 percent by weight. The liposome is typically comprised of a phospholipid bilayer, which may contain phospholipids which have a hydrophobic tail and a hydrophilic head, wherein the tails face one another and form a bilayer, and once enough phospholipids aggregate, a micelle forms, and when even more aggregate, a phospholipid bilayer forms. Many phospholipids contain a diglyceride, a phosphate group, and a simple organic molecule such as choline. The liposome has a hollow cavity which easily allows an aqueous or water soluble molecule to become encapsulated. Using any solvent that is polar enough to not dissolve the lipids achieves this result, allowing the lipid to encapsulate various types of solvent and drugs which are soluble in the solvent used. Once inside, an aqueous or water soluble molecule is effectively trapped because it cannot pass through the fatty portion of the liposome created by the diglycerides. The liposome can be combined with other components to release the drugs upon binding via a peptide to a targeted cell.

FIG. 1 is a cross-sectional view of one embodiment of a liposome. As shown in FIG. 1, the phospholipids preferably comprise of hydrophobic tails 105, hydrophilic heads 110, and a hollow cavity 115. Preferably, the hydrophobic tails 105 aggregate towards one another, leaving hydrophilic heads 110 to interact with the aqueous solution. Preferably this causes the hollow cavity 115 to form.

The therapeutic drug is a component added to kill cancerous cells and tumors. Preferably, the therapeutic drug is in an amount up to 55.0 percent by weight, and more preferably between 8.0 and 50.0 percent by weight.

The gemcitabine component of the compound typically has the ability to treat, but is not limited to, non-small cell lung cancer, pancreatic cancer, bladder cancer, and breast cancer. It is being investigated for use in esophageal cancer, and is used experimentally in lymphomas and various other tumors. Preferably, the gemcitabine component is in an amount up to 55.0 percent by weight, and more preferably between 8.0 and 50.0 percent by weight.

The gemcitabine component of the compound typically has the formula C₉H₁₁F₂N₃O₄ with the following structure:

[chemical structure]

The cisplatin component of the compound typically has the ability to treat, but is not limited to, sarcomas, some carcinomas (e.g. small cell lung cancer, and ovarian cancer), lymphomas, and germ cell tumors. Cisplatin is particularly effective against testicular cancer. Preferably, the cisplatin component is in an amount up to 55.0 percent by weight, and more preferably between 8.0 and 50.0 percent by weight.

The cisplatin component of the compound typically has the formula H₆Cl₂N₂Pt with the following structure:

[chemical structure]

The doxorubicin component of the compound typically has the ability to treat, but is not limited to, some leukemias and Hodgkin's lymphoma, as well as cancers of the bladder, breast, stomach, lung, ovaries, thyroid, soft tissue sarcoma, multiple myeloma, and others. Preferably, the doxorubicin is in an amount up to 55.0 percent by weight, and more preferably between 8.0 and 50.0 percent by weight.

The doxorubicin component of the compound typically has the formula C₂₇H₂₉NO₁₁ with the following structure:

[chemical structure]

The Collagen binding peptide is a peptide which functions to direct the compound towards cancer and tumors. The peptide has a higher affinity for cancerous cells and tumors than regular cells, which allows the compound to bind to a cancerous cell or tumor and release the encapsulated drug near the cancerous cell or tumor for an increased uptake rate for cancerous cells or tumors. Preferably the peptide component is in an amount up to 20.0 percent by weight, and more preferably between 8.0 to 16.0 percent by weight. One particular preferred embodiment contains about 5.13 percent by weight based on the total weight percent of the composition.

The peptide typically has the formula with the sequence: Trp-Arg-Glu-Pro-Ser-Phe-Met-Ala-Leu-Ser (WREPSFMALS).

The DSPE-PEG is added to stabilize the compound while it is in the bloodstream. Because the liposome is fatty, it has a low solubility in water, which is the main component of a bloodstream. DSPE-PEG is able to attach via the peptide, and provide a hydrophilic PEG shell, which allows the compound to be more soluble, and as a result, stay in the blood stream for longer. If it is in the bloodstream for longer, it has more chances to bind to cancerous cells and tumors. PEG is a repeated molecular subunit and can have anywhere from 10 to 10,000 repeats.

The DSPE-PEG component of the polish composition typically has the formula (DSPE) C₄₁H₈₂NO₈P (PEG) C₂ₙH₄ₙ₊₂Oₙ₊₁ with the following structure:

[chemical structure]

PEG has the following structure:

[chemical structure]

It should also be understood that other known specific instances of compounds such as liposomes, therapeutic drugs, peptides, and buffers are usable without departing from the spirit of the invention. Examples include but are not limited to:

Effective amounts of one or more therapeutic drugs may be added to impart the ability to fight cancers both treated by the claimed drugs and cancers not explicitly treated by the claimed drugs. Effective amounts of one or more therapeutic drugs typically means an amount of therapeutic drugs which kills more cancerous cells, regardless of increased damage to non-cancerous cells. The ratio of targeted cancerous cells to non-cancerous cells is considered in the effectiveness of the peptides.

Effective amounts of one or more peptides may be added to impact the ability to guide the entire compound to another type of cell. An effective amount of one or more peptides means that the peptides increase the compound's binding affinity for cancerous cells.

Although specific compounds are listed in Table 1, it should be understood that equivalent compounds may be used including, but not limited to, unlisted liposomes, other therapeutic drugs, other particularly binding peptides, and other agents that are known to increase the half-life and effectiveness of drugs. Effectiveness of drugs increases as cancerous cells are killed.

The cancer fighting compound of the present invention may be made through a series of chemical reactions. Because the specific mechanics of the reactions used are known to an individual of ordinary skill in the art, specifics may be left out because addition of unnecessary facts does not increase one's understanding of the invention or the process for making it.

The first step is to create the Peptide-PEG-DSPE conjugate. This is done by mixing NHS-PEG-DSPE with the desired peptides, in Phosphate Buffer Solution (PBS) at pH 7.4 for 24 hours. This creates the Peptide-PEG-DSPE conjugate. NHS-PEG-DSPE is well known in the art and can be commercially purchased or very easily manufactured.

The second step is to form the liposome. The Peptide-PEG-DSPE conjugate is combined in a lipid solution consisting of lipid and lipid-PEG conjugate, which creates a lipid film. This then forms a multilamellar vesicle and after extrusion, it creates an Empty Decorated Liposome. An Empty Decorated Liposome is a liposome which has a Peptide-PEG-DSPE conjugate attached to it.

In order to add therapeutic molecules to the compound, therapeutic molecules are introduced to the solution prior to formation of the lipid film. The concentration of therapeutic molecules used determines the amount and concentration of therapeutic molecules in the final compound.

The following Table 3 represents a preferred embodiment of the liposome and DSPE-PEG components of the cancer fighting compound. This embodiment is not the sole combination of usable parts, and it is understood that variations from these percentages may be used as well. Unless otherwise noted, all parts, percentages, and ratios reported in the following table are on a molar percentage basis, and all reagents and compounds used in the table were obtained, or are available, from chemical suppliers, or may be synthesized by conventional techniques.

TABLE 3

| Ingredient | Preferred Molar % |
|---|---|
| DPPC - 1,2-dipalmitoyl-sn-glycero-3- | 62% |
| phosphocholine | |
| Cholesterol | 32% |
| Reactive Lipid DSPE-PEG | 6% |

The following table 4 represents the same information as in Table 3, except that this information is presented in percent by weight amounts, and all reagents and compounds used in the table were obtained, or are available, from chemical suppliers, or may be synthesized by conventional techniques.

TABLE 4

| Ingredient | Preferred Weight % |
|---|---|
| DPPC - 1,2-dipalmitoyl-sn-glycero-3- | 59.9% |
| phosphocholine | |
| Cholesterol | 16.3% |
| Reactive Lipid DSPE-PEG | 23.8% |

Although tumors are the typical target of the current cancer fighting compound, it should be understood that the present invention may be used to fight cancers, cancerous cells, and tumors.

Using the compound or method of the present invention allows a user to have another option in fighting cancerous cells or a tumor. The life of a patient may be extended if the treatment goes well.

The foregoing description of the preferred embodiment of the invention has been presented for the purposes of illustration and description. While multiple embodiments are disclosed, still other embodiments of the present invention will become apparent to those skilled in the art from the above detailed description, which shows and describes illustrative embodiments of the invention. As will be realized, the invention is capable of modifications in various obvious aspects, all without departing from the spirit and scope of the present invention. Accordingly, the detailed description is to be regarded as illustrative in nature and not restrictive. Also, although not explicitly recited, one or more embodiments of the invention may be practiced in combination or conjunction with one another. Furthermore, the reference or non-reference to a particular embodiment of the invention shall not be interpreted to limit the scope the invention. It is intended that the scope of the invention not be limited by this detailed description, but by the claims and the equivalents to the claims that are appended hereto.

What is claimed is:

1. A cancer treatment compound comprising:
one or more therapeutic agents in an amount of from 9.0 to 50.0 percent by weight based on a total weight of said cancer fighting compound;
a liposome component in an amount of from 38.0 to 70.0 percent by weight based on a total weight of said cancer fighting compound.

2. The cancer treatment compound of claim 1, wherein said one or more therapeutic agents are encapsulated in said liposome.

3. The cancer treatment compound of claim 2, wherein said liposome component comprises a phospholipid bilayer;

4. The cancer treatment compound of claim 3, further comprising a DSPE-PEG component in an amount from 12.0 to 21.0 percent by weight based on a total weight of said cancer fighting compound; and
wherein said one or more therapeutic agents is doxorubicin.

5. The cancer treatment compound of claim 3, further comprising:
a peptides component in an amount of from 8.0 to 16.0 percent by weight based on a total weight of said cancer fighting compound; and
a DSPE-PEG component in an amount of from 10.0 to 20.0 percent by weight based on a total weight of said cancer fighting compound;

6. The cancer treatment compound of claim 5, wherein said peptide component is attached to said liposome component.

7. The cancer treatment compound of claim 6, wherein said DSPE-PEG component is attached to said peptide component.

8. The cancer treatment compound of claim 7, wherein said peptides component causes said cancer treatment compound to preferentially bind to tumors, and thereby accumulate at tumors;
wherein said cancer treatment compound releases said one or more therapeutic agents upon binding with tumors.

9. The cancer treatment compound of claim 8, wherein said DSPE-PEG component stabilizes said cancer treatment compound to allow said cancer treatment compound to spend more time in a bloodstream before becoming less effective.

10. The cancer treatment compound of claim 9, wherein said one or more therapeutic agents is selected from one or more of the following classes: anti-neoplastic therapeutic agents, non-steroidal anti-inflammatory therapeutic agents, steroidal anti-inflammatory therapeutic agents, nucleic acid or nucleotide sequence for non-viral gene therapy, hormones, peptides, growth factors, antiangiogenic compounds, antisense oligonucleotides, anti-micro RNA molecules, microRNA molecules, herbal drugs, and antibodies.

11. The cancer treatment compound of claim 10, wherein said one or more therapeutic agents is a cancer fighting drug selected from one or more of the following: gemcitabine, cisplatin, and doxorubicin.

12. The cancer treatment compound of claim 10 or 11, wherein said peptide component is a Collagen binding peptide.

13. A cancer treatment compound comprising:
one or more therapeutic agents in an amount of from 8.0 to 48.0 percent by weight based on a total weight of said cancer fighting compound;
a liposome component in an amount of from 34.0 to 63.0 percent by weight based on a total weight of said cancer fighting compound;
a peptides component in an amount of from 8.0 to 16.0 percent by weight based on a total weight of said cancer fighting compound;
a DSPE-PEG component in an amount of from 12.0 to 21.0 percent by weight based on a total weight of said cancer fighting compound;
wherein said one or more therapeutic agents is selected from one or more of the following classes: anti-neoplastic therapeutic agents, non-steroidal anti-inflammatory therapeutic agents, steroidal anti-inflammatory therapeutic agents, nucleic acid or nucleotide sequence for non-viral gene therapy, hormones, peptides, growth factors, antiangiogenic compounds, antisense oligonucleotides, anti-micro RNA molecules, microRNA molecules, herbal drugs, and antibodies;
wherein said peptides component is a Collagen binding peptide;
wherein said one or more cancer fighting drugs is encapsulated in said liposome component;
wherein said liposome component comprises a phospholipid bilayer;
wherein said peptides component attach to said liposome component, and causes said cancer treatment compound to preferentially bind to tumors;
wherein said cancer treatment compound releases said one or more cancer fighting drugs upon binding with cancerous cells and tumors;
wherein said DSPE-PEG component is attached to said peptide component, and stabilizes said cancer treatment compound to allow said cancer treatment compound to spend more time in a bloodstream before becoming less effective.

14. The cancer treatment compound of claim 13, wherein said one or more therapeutic agents is selected from one or more of: gemcitabine, cisplatin, and doxorubicin.

15. The cancer treatment compound of claim 14, wherein said Collagen binding peptide is selected from the group of the peptide sequences consisting of:

| | |
|---|---|
| Seq1: | Trp-Arg-Glu-Pro-Ser-Phe-Met-Ala-Leu-Ser |
| | (WREPSFMALS); |
| Seq2: | Trp-Arg-Glu-Pro-Ser-Phe-Cys-Ala-Leu-Ser |
| | (WREPSFCALS); |
| Seq3: | Trp-Arg-Asp-Pro-Ser-Phe-Met-Ala-Leu-Ser |
| | (WRDPSFMALS); |
| Seq4: | Trp-Arg-Asp-Pro-Ser-Phe-Cys-Ala-Leu-Ser |
| | (WRDPSFCALS); |
| Seq5: | Trp-Arg-Glu-Pro-Ser-Phe-Met-Ala-Ile-Ser |
| | (WREPSFMAIS); |
| Seq6: | Trp-Arg-Glu-Pro-Ser-Phe-Cys-Ala-Leu-Ser |
| | (WREPSFCALS); |
| Seq7: | Trp-Arg-Asp-Pro-Ser-Phe-Met-Ala-Leu-Ser |
| | (WRDPSFMALS); |
| and | |
| Seq8: | Trp-Arg-Glu-Pro-Ser-Phe-Cys-Ala-Leu-Ser |
| | (WREPSFCALS). |

16. The cancer treatment compound of claim 15, wherein said Collagen binding peptides may have a fluorescent dye attached at either end of the peptide by addition of an amino acid sequence PPGP to the front of said Collagen binding peptide.

17. The cancer treatment compound of claim 16 wherein said Collagen binding peptides have PPGP added to the back end, allowing for tandem repeats of the peptide.


SEQUENCE LISTING (17 sequences)

SEQ ID NO: 1 - protein, 10 aa, Artificial Sequence
  This peptide is chemically synthesized by a company in North Carolina called PEPTIDE 2.0.
WREPSFMALS

SEQ ID NO: 2 - protein, 10 aa, Artificial Sequence
  This peptide is chemically synthesized by a company in North Carolina called PEPTIDE 2.0.
WREPSFCALS

SEQ ID NO: 3 - protein, 10 aa, Artificial Sequence
  This peptide is chemically synthesized by a company in North Carolina called PEPTIDE 2.0.
WRDPSFMALS

SEQ ID NO: 4 - protein, 10 aa, Artificial Sequence
  This peptide is chemically synthesized by a company in North Carolina called PEPTIDE 2.0.
WRDPSFCALS

SEQ ID NO: 5 - protein, 10 aa, Artificial Sequence
  This peptide is chemically synthesized by a company in North Carolina called PEPTIDE 2.0.
WREPSFMAIS

SEQ ID NO: 6 - protein, 10 aa, Artificial Sequence
  This peptide is chemically synthesized by a company in North Carolina called PEPTIDE 2.0.
WREPSFCALS

SEQ ID NO: 7 - protein, 10 aa, Artificial Sequence
  This peptide is chemically synthesized by a company in North Carolina called PEPTIDE 2.0.
WRDPSFMALS

SEQ ID NO: 8 - protein, 10 aa, Artificial Sequence
  This peptide is chemically synthesized by a company in North Carolina called PEPTIDE 2.0.
WREPSFCALS

SEQ ID NO: 9 - protein, 14 aa, Artificial Sequence
  This peptide is chemically synthesized by a company in North Carolina called PEPTIDE 2.0.
PPGPWREPSFMALS

SEQ ID NO: 10 - protein, 19 aa, Artificial Sequence
  This peptide is chemically synthesized by a company in North Carolina called PEPTIDE 2.0.
GPPGPWREPSFMALSGPPG

SEQ ID NO: 11 - protein, 19 aa, Artificial Sequence
  This peptide is chemically synthesized by a company in North Carolina called PEPTIDE 2.0.
GPPGPWREPSFCALSGPPG

SEQ ID NO: 12 - protein, 19 aa, Artificial Sequence
  This peptide is chemically synthesized by a company in North Carolina called PEPTIDE 2.0.
GPPGPWRDPSFMALSGPPG

SEQ ID NO: 13 - protein, 19 aa, Artificial Sequence
  This peptide is chemically synthesized by a company in North Carolina called PEPTIDE 2.0.
GPPGPWRDPSFMALSGPPG

SEQ ID NO: 14 - protein, 19 aa, Artificial Sequence
  This peptide is chemically synthesized by a company in North Carolina called PEPTIDE 2.0.
GPPGPWREPSFMAISGPPG

SEQ ID NO: 15 - protein, 19 aa, Artificial Sequence
  This peptide is chemically synthesized by a company in North Carolina called PEPTIDE 2.0.
GPPGPWREPSFCAISGPPG

SEQ ID NO: 16 - protein, 19 aa, Artificial Sequence
  This peptide is chemically synthesized by a company in North Carolina called PEPTIDE 2.0.
GPPGPWRDPSFMAISGPPG

SEQ ID NO: 17 - protein, 19 aa, Artificial Sequence
  This peptide is chemically synthesized by a company in North Carolina called PEPTIDE 2.0.
GPPGPWRDPSFCAISGPPG